(19) United States
(12) Patent Application Publication
(10) Pub. No.: US 2016/0175140 A1
(43) Pub. Date: Jun. 23, 2016

(54) PORTABLE THERMAL THERAPY APPARATUS AND METHOD THEREFOR

(71) Applicant: SmartAnt Telecom Co., Ltd., Hsinchu (TW)

(72) Inventors: Yuan-Lien CHEN, Taichung (TW); Po-Chuan HUANG, Hsinchu (TW); Zhao-Nan LIU, Miaoli County (TW); Wei-Jung CHEN, Tainan (TW); Tzyy-Huei LEE, Taipei (TW); Po-Wei LIN, Taipei (TW)

(21) Appl. No.: 14/573,742

(22) Filed: Dec. 17, 2014

(51) Int. Cl. A61F 7/00; G05B 19/042

(52) U.S. Cl. CPC A61F 7/007; G05B 19/0423; A61F 2007/008; A61F 2007/0094; G05B 2219/23169

(57) ABSTRACT

A portable thermal therapy apparatus is for executing an operating method capable of recording the thermal therapy procedure. The operating method for a portable thermal therapy apparatus includes receiving an operating command, the operating command at least having an operating parameter, executing an operating procedure according to the operating command, the operating procedure at least including an operating mode, and recording the operating mode into an operating log message.

### BACKGROUND OF THE INVENTION

1. Technical Field of the Invention

The present invention relates to a portable thermal therapy apparatus and a method thereof, particularly to a portable thermal therapy apparatus and a method thereof able to receive operating commands and record operating log messages.

2. Description of the Related Art

As time progresses, modern people have more and more pressure in their work and daily life and lack of sufficient exercise, so people often suffer from musculoskeletal pain. However, in order to ease the pain, there is a variety of treatments to choose from, such as electrotherapy, massage, thermal therapy . . . etc.

With regard to thermal therapy, it is a treatment of using a heat source on a human body for penetrating into the subcutaneous tissue to the deep muscle, so that the effect of easing and eliminating the pain is achieved. However, in order to allow the heat to penetrate into different depths of the human body, the heater is used to generate a heat source for the upper part of the human body to achieve the effect of thermal therapy.

However, generally diathermy is adopted for the effect of deep thermal therapy in the deeper part of the human body. Diathermy is a treatment of heating the human tissue. In other words, diathermy uses RF (radio frequency) electromagnetic waves to irradiate the human body, and the electromagnetic waves create the resonance in the deep part of the human muscles for generating heat, so that the effect of deep thermal therapy is achieved.

Common thermal therapy devices are large stationary machine and needed to be placed in the medical institutions. On the other hand, the setup process of the thermal therapy is complex, and ordinary users can not easily setup and use the device. Therefore, patients must use the device with the assistance of professional medical staffs for the complex setup.

### SUMMARY OF THE INVENTION

According the problem, the present invention provides a portable thermal therapy apparatus and a method thereof able to receive operating commands, record operating log messages, and recording the usage status.

The operating method for a portable thermal therapy apparatus of the present invention includes receiving an operating command, the operating command at least having an operating parameter, executing an operating procedure according to the operating command, the operating procedure at least including an operating mode, and recording the operating mode into an operating log message.

The portable thermal therapy apparatus of the present invention includes an input unit, a processor and a memory unit, a power management unit, a thermal therapy module, a battery, and an external power source. The input unit is for receiving an operating command and the operating command at least includes an operating parameter. The processor and a memory unit is for executing an operating procedure according to the operating command and recording the operating mode into an operating log message, and the operating procedure at least includes an operating mode. The power management unit is for executing a charging procedure. The thermal therapy module is for executing a running procedure. The battery is for providing the portable thermal therapy apparatus with necessary electricity. The external power source is for providing a direct current (DC) power source to the power management unit of the portable thermal therapy apparatus.

In an embodiment of the present invention, the step of receiving an operating command, the operating command at least having an operating parameter, includes receiving an input command, determining whether the operating mode is an input mode, and executing the following steps when the operating mode is the input mode: determining whether an input item is the operating parameter, executing a writing operating parameter procedure when the input item is the operating parameter, and executing the operating mode.

In an embodiment of the present invention, the step of recording the operating mode into the operating log message includes writing the working time parameter, the working cycle parameter of the short wave module, the working period parameter, and the operated time into a memory.

In summary, the portable thermal therapy apparatus of the present invention receives the operating command, and the operating command includes at least an operating parameter. The operating procedure is executed according to the operating command, and the operating procedure at least includes the operating mode. The working module records the operating mode into the operating log message to the memory. Therefore, the portable thermal therapy apparatus of the present invention provides portability, loading the therapy in advance, and the function of recording operating log message, so that the portable thermal therapy apparatus is more convenient for the user.

The contents of the present invention set forth and the embodiments hereinafter are for demonstrating and illustrating the spirit and principles of the present invention, and for providing further explanation of the claims.

### BRIEF DESCRIPTION OF THE DRAWINGS

The present invention will become more fully understood from the detailed description given hereinbelow and the accompanying drawings, which are given by way of illustration only and thus are not limitative of the present invention and wherein:

FIG. 1 is a structural diagram of a system according to an embodiment of the present invention.

FIG. 2 is a structural diagram of a thermal therapy module according to an embodiment of the present invention.

FIG. 3 is a flowchart of an operating method according to an embodiment of the present invention.

FIG. 4 is a flowchart of a system operation according to an embodiment of the present invention.

FIG. 5 is a flowchart of the step S330 according to an embodiment of the present invention.

FIG. 6 is a flowchart of the step S360 according to an embodiment of the present invention.

FIG. 7 is a flowchart of the step S340 according to an embodiment of the present invention.

FIG. 8 is a flowchart of the step S344 according to an embodiment of the present invention.

FIG. 9A is a flowchart of part of the step S348 according to an embodiment of the present invention.

FIG. 9B is a flowchart of part of the step S348 following FIG. 9A according to an embodiment of the present invention.

FIG. 10A is a flowchart of part of the step S350 according to an embodiment of the present invention.

FIG. 10B is a flowchart of part of the step S350 following FIG. 10A according to an embodiment of the present invention.

FIG. 11 is a flowchart of the step S230 according to an embodiment of the present invention.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

The present invention provides a portable thermal therapy apparatus and the method thereof, wherein the method is implemented for the portable thermal therapy apparatus.

For more specific explanation, the portable thermal therapy apparatus according to an embodiment of the present invention is explained with a heating cover band (not illustrated in the figure) for a human body, such as the knees. The heating cover band has a sticky buckle belt and a user buckles the knees with the heating cover band, and the heating cover band generates heats to ease the uncomfortableness of the knees. The heating cover band for the knees is with a battery for the user to carry or use during exercise, so that the portability is achieved.

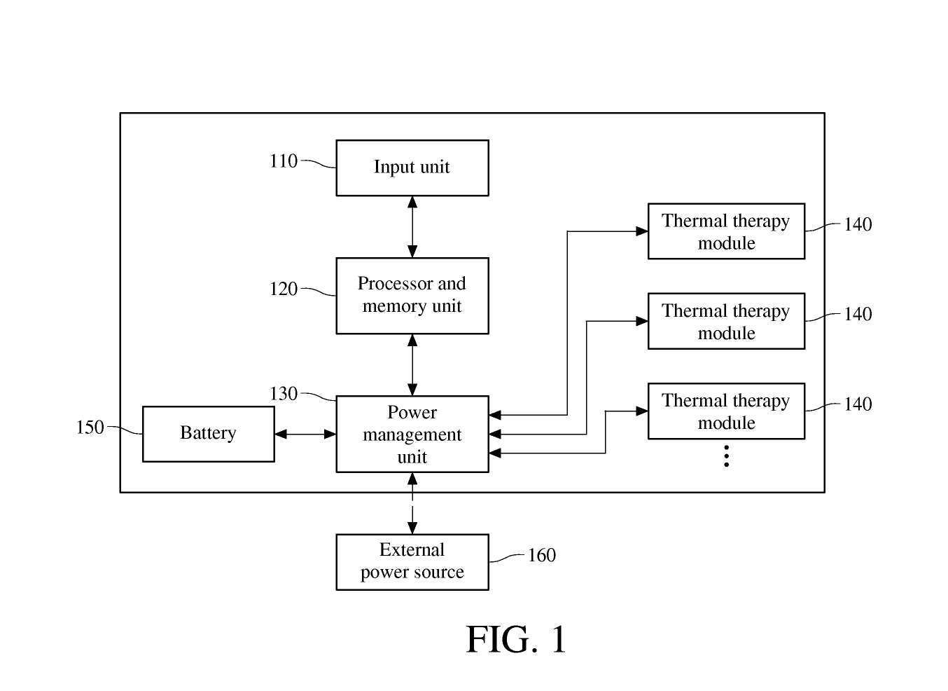

Please refer to FIG. 1. FIG. 1 is a structural diagram of a system according to an embodiment of the present invention. As shown in FIG. 1, the portable thermal therapy apparatus includes an input unit 110, a processor and a memory unit 120, a power management unit 130, a thermal therapy module 140, a battery 150, and an external power source 160. The input unit 110 is for receiving an operating command inputted by an external user. In the present embodiment, the operating command includes an operating parameter and a system parameter, wherein the operating parameter is a set of parameters related to the thermal therapy, and the system parameter includes a number of usage parameter and a clock parameter. The input unit 110 is a terminal interface connected with an external computer or an external controller. For example, the input unit 110 includes but not limited to a USB (Universal Serial Bus), a RS-232, or any other transmission interface for receiving input data.

The processor and the memory unit 120 are for executing an operating procedure according to the operating command, and the operating procedure at least includes an operating mode. In the present embodiment, the operating procedure includes an input mode and an output mode. The input mode stands for the related procedures for the system to receive external commands, and the output mode stands for the procedures for the system to output the recorded information inside the system. For example, when the system is in the input mode, the user enters related settings. When the system is in output mode, the user obtains the related information from the device. The processor mainly executes the programs related to the operating method of the present invention, and the memory unit is for recording the operating mode into an operating log message. The operating log message is but not limited to a text file or a data type. For example, the system outputs the log message into a file, so that the user is able to examine the therapy on a computer.

The power management unit 130 is for executing a charging procedure. The thermal therapy module 140 is for executing a running procedure. The battery 150 provides the electricity for the portable thermal therapy apparatus to operate. For example, the battery 150 is but not limited to a lithium battery, an alkaline battery, or any other power source. The external power source 160 is for providing a direct current (DC) power source to the power management unit 130 of the portable thermal therapy apparatus, such as a DC charger. The related devices and procedures are mentioned later.

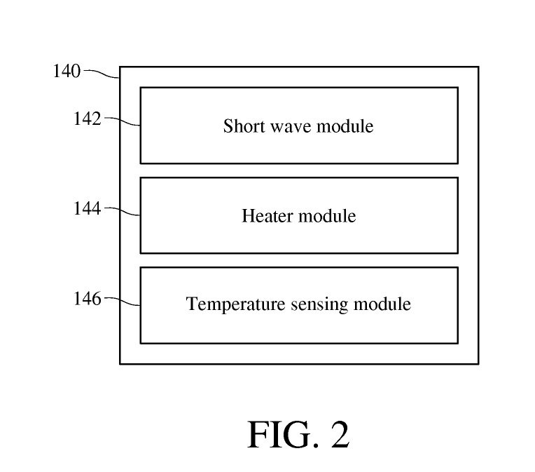

Please refer to FIG. 2. FIG. 2 is a structural diagram of a thermal therapy module according to an embodiment of the present invention. As shown in FIG. 2, the thermal therapy module 140 includes a short wave module 142 for generating a diathermy, a heater module 144 for generating a heat source, and a temperature sensing module 146 for monitoring the heater module 144. For example, diathermy is a therapy of using the heat effect generated by short wave, and the short wave is a kind of electromagnetic wave. Diathermy is a kind of deep heat thermal therapy (the thermal therapy depth is more than 2 millimeters in general medical science) of the physical therapy. The short wave module 142 is but not limited to the device for generating the short wave frequency of 27.12 MHz.

Please refer to FIG. 1 again. The input unit 110, the process or memory unit 120 or the power management unit 130 are called a working module in the following explanations. The working module executes the step S210 to the step S230 to receive the operating command, to execute in at least one operating mode, and finally to record the operating mode into an operating log message. For example, the input unit 110 is but not limited to a touch screen.

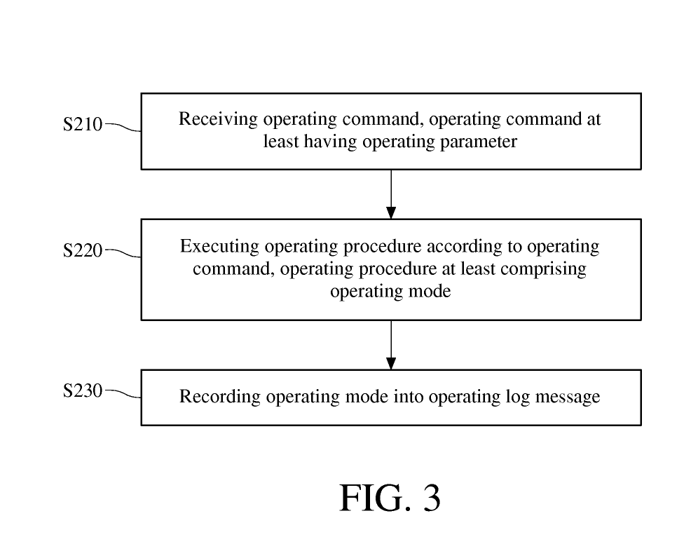

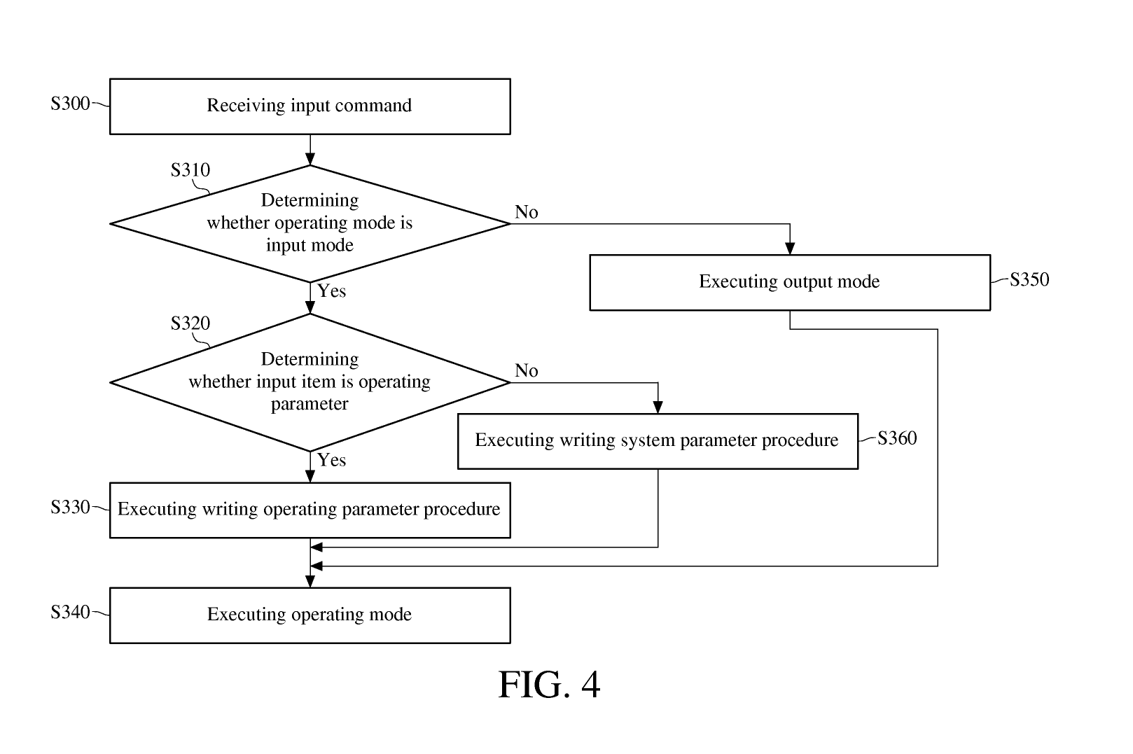

Please refer to FIG. 3. FIG. 3 is a flowchart of an operating method according to an embodiment of the present invention. As shown in FIG. 3, the working module first receives the operating command, and the operating command at least includes the operating parameter, as shown in the step S210. The details of the step S210 is further described in the FIG. 4. FIG. 4 is a flowchart of a system operation according to an embodiment of the present invention. The working module receives an input command from a user by keying or a computer. Then the working module determines whether the operating mode is the input mode (the step S310), and when the operating mode is the input mode, the following steps are executed. The working module determines whether an input item is the operating parameter (the step S320). In the present embodiment, the input item is but not limited to the item for inputting, such as the operating parameters which are further introduced later. When the input item is the operating parameter, it indicates that the user inputs the operating parameter related to the thermal therapy to the portable thermal therapy apparatus, a writing operating parameter procedure is executed for writing the operating command into the memory unit of the working module (the step S330). Last, the operating mode is executed (the step S340) to begin the main thermal therapy procedure of the portable thermal therapy apparatus.

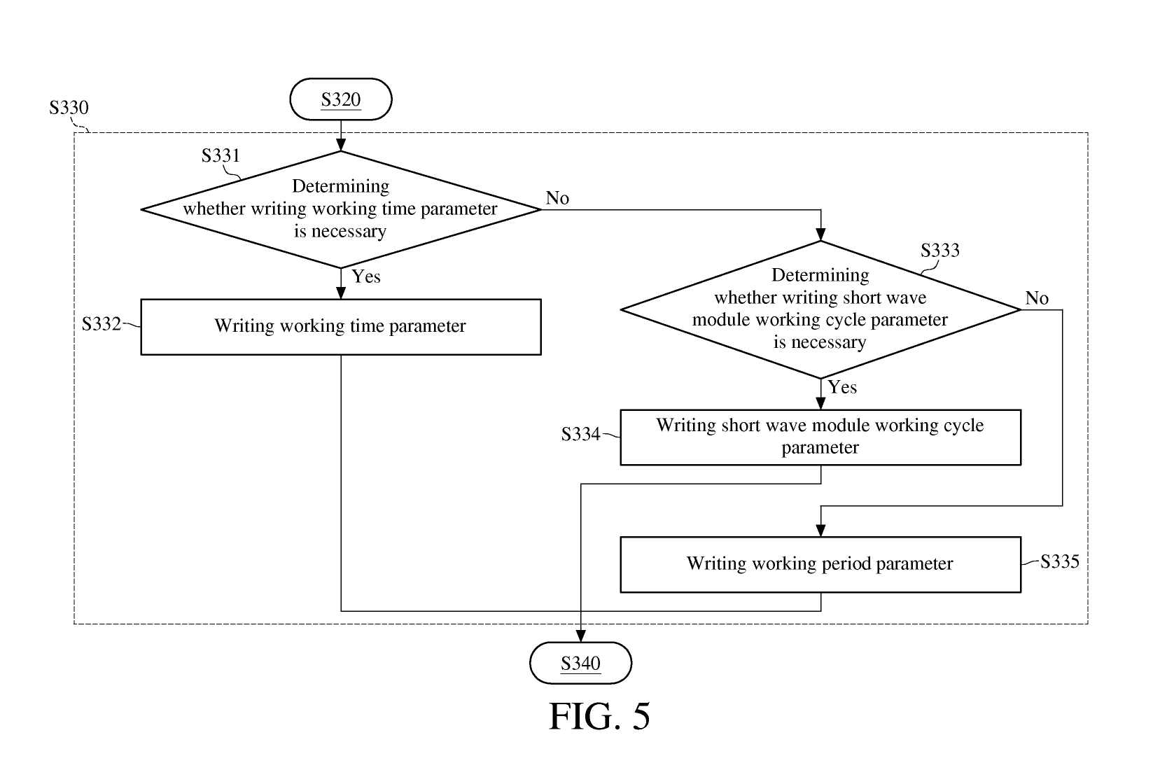

Please refer to FIG. 5. FIG. 5 is a flowchart of the step S330 according to an embodiment of the present invention. The writing operating parameter procedure includes the following steps. The working module determines whether writing a working time parameter is necessary (the step S331). The working time parameter is written when writing the working time parameter is necessary (the step S332). In the present embodiment, the working time parameter is a time unit of turning on a heater thermal therapy procedure and a diathermy procedure. For example, the device executes the heater thermal therapy procedure for 5 minutes and then executes the diathermy procedure for 10 minutes, or the device executes the heater thermal therapy procedure for 10 minutes and then executes the diathermy procedure for 5 minutes. The present invention does not have any limitation. The following steps are executed when writing the working time parameter is not necessary. The working module determines whether writing a short wave module working cycle parameter is necessary (the step S333). The short wave module working cycle parameter is written when writing the short wave module working cycle parameter is necessary (the step S334). In the present embodiment, the short wave module working cycle parameter represents that the frequency of short wave module 142 being turned on and off. For example, during the operation, the short wave module 142 is turned off every ⅙ sec. The present invention does not have any limitation. The working period parameter is written when writing the short wave module working cycle parameter is not necessary (the step S335). In the present embodiment, taking a therapy with 8 working period parameters for example, the working period parameters are the combination of a plurality of different operating parameters, so the working period parameter stands for turning on two of the working time parameter and turning off 4 of the working time parameter and then turning on 2 of the working time parameter, or for executing all of the 8 working time parameters. The present invention does not have any limitation. When the step 331 to the step 335 are executed by the working module, the system records the necessary procedure parameter related to the thermal therapy for the user, and then the system executes the thermal therapy procedure in the operating mode according to these related procedure parameter.

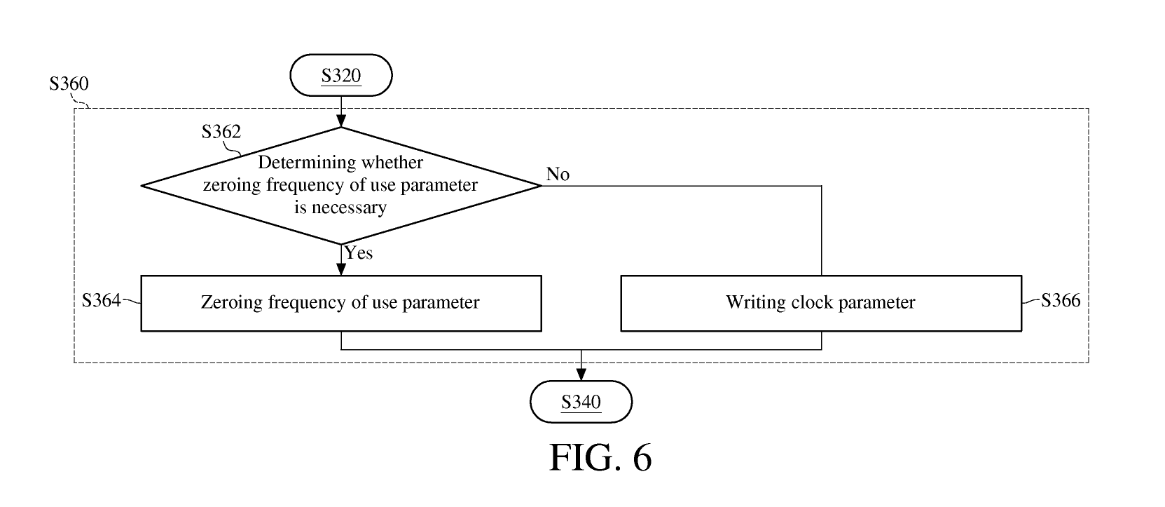

When the working module determines that the input item is not the operating parameter, the working module executes the writing system parameter procedure. Please refer to FIG. 6. FIG. 6 is a flowchart of the step S360 according to an embodiment of the present invention. The writing system parameter procedure includes the following steps: the working module determines whether zeroing a frequency of use parameter is necessary (the step S362). In the present embodiment, the frequency of use parameter is for recording the number of usage of the portable thermal therapy apparatus by the user. Therefore, the frequency of use parameter is zeroed when zeroing the frequency of use parameter is necessary (the step S364). Zeroing the frequency of use parameter represents that a frequency of use parameter of a unit is set to zero for the user to count the numbers of thermal therapy from different users. When zeroing the frequency of use parameter is not necessary, a clock parameter is written (the step S366). The clock parameter is a parameter of time format, and the user inputs a time parameter to the clock parameter of the system to setup or correct the system time of the portable thermal therapy apparatus.

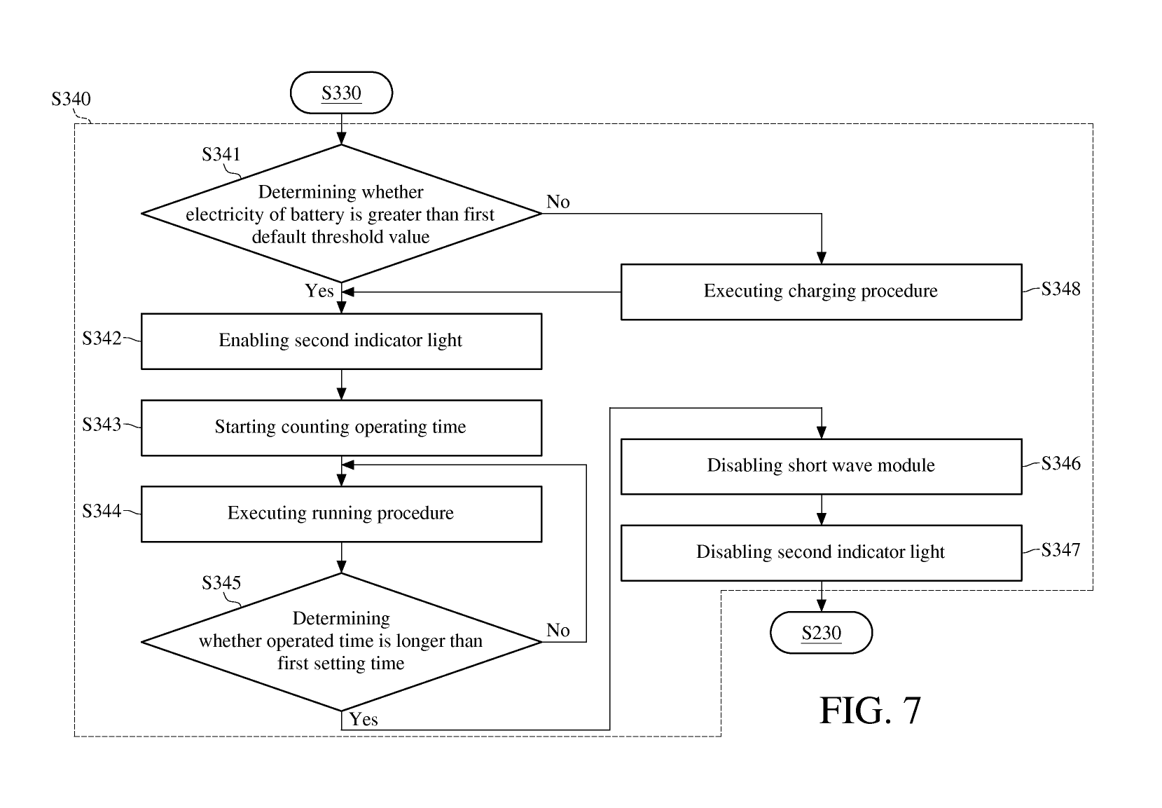

Finally, the working module executes the operating mode according to the operating parameters in the memory unit (the step S340). Please refer to FIG. 7. FIG. 7 is a flowchart of the step S340 according to an embodiment of the present invention. The working module determines whether the electricity of the battery 150 is greater than a first default threshold value (the step S341). When the electricity of the battery 150 is greater than a first default threshold value, the working module executes the following steps. First, a second indicator light is enabled (the step S342). In the present embodiment, the first default threshold value is but not limited to half of the voltage value of the total electricity in the battery 150. The operation indicator light is but not limited to a LED (Light-Emitting Diode). Next, an operated time is started to count (the step S343). The operated time is for counting the operated time of the portable thermal therapy apparatus to provide the system or the user the used time of the portable thermal therapy apparatus. For example, when the doctor suggests the user to use the portable thermal therapy apparatus for 15 minutes during the therapy, the user obtains the operated time for checking the duration of the treatment.

Next, a running procedure is executed (the step S344). The detail of the running procedure is further described later. Then, the working module determines whether an operated time is longer than a first setting time (the step S345). When the operated time is longer than the first setting time, the working module executes the following steps: disabling a short wave module (the step S346) and disabling the second indicator light (the step S347). Last, the working module executes recording the operating mode into an operating log message (the step S230). For example, when the operated time is less than or equal to the first setting time, the system keeps executing the running procedure, and when the operated time is greater than the first setting time, it means that the thermal therapy procedure is finished, and the system executes the step S230 to record the related information of the therapy.

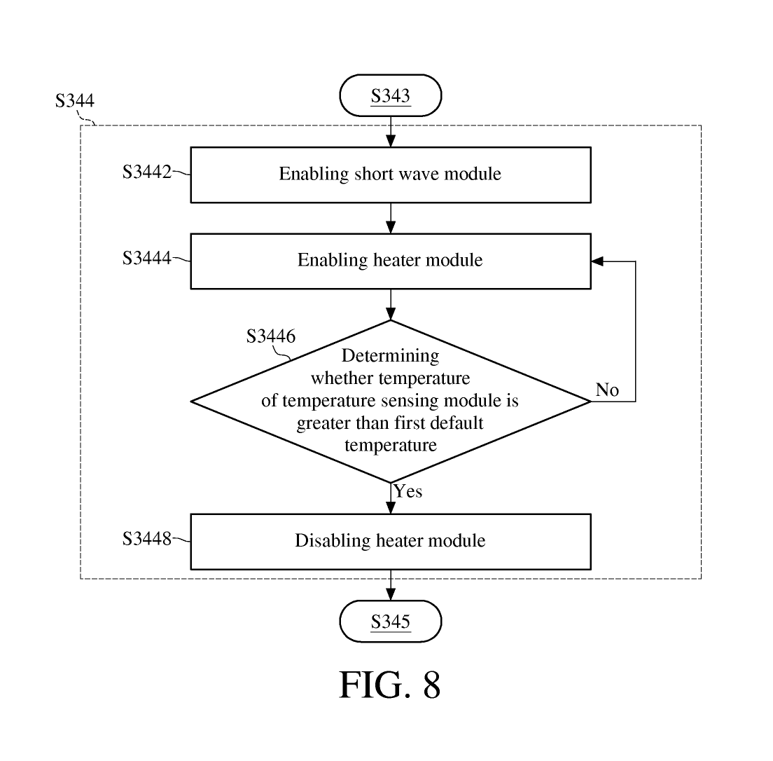

Please refer to FIG. 8. FIG. 8 is a flowchart of the step S344 according to an embodiment of the present invention. The flowchart mainly describes the operating process of the thermal therapy module 140 of the portable thermal therapy apparatus. The thermal therapy module 140 includes the short wave module 142, the heater module 144, and the temperature sensing module 146. The short wave module 142 is for receiving the commands of the working module to generate RF (radio frequency) electromagnetic wave. The RF electromagnetic wave penetrates the inner muscles of the human body to generate heats. The heater module 144 is but not limited to an infrared light, a heater coil, or a heating pad.

The temperature sensing module 146 monitors the heater module 144, so that the heater module 144 operates in a first default temperature. The working module first enables the short wave module 142 (the step S3442) and next enables the heater module 144 (the step S3444), and determines whether the temperature of the temperature sensing module 146 is greater than the first default temperature (the step S3446). When the temperature of the temperature sensing module 146 is greater than the first default temperature, the heater module 144 is disabled (the step S3448). In other words, when the temperature of the heater module 144 is less than or equal to the first default temperature, the working module continuously enables the heater module 144. When the temperature of the heater module 144 is greater than the first default temperature, the heater module 144 is disabled, so that a constant temperature is achieved. For example, the first default temperature is 40° C. and when the temperature of the heater module 144 is less than or equal to 40° C., the working module continuously enables the heater module 144 to increase the temperature. When the temperature of the heater module 144 is greater than the first default temperature, the heater module 144 is disabled, so that the device maintains 40° C.

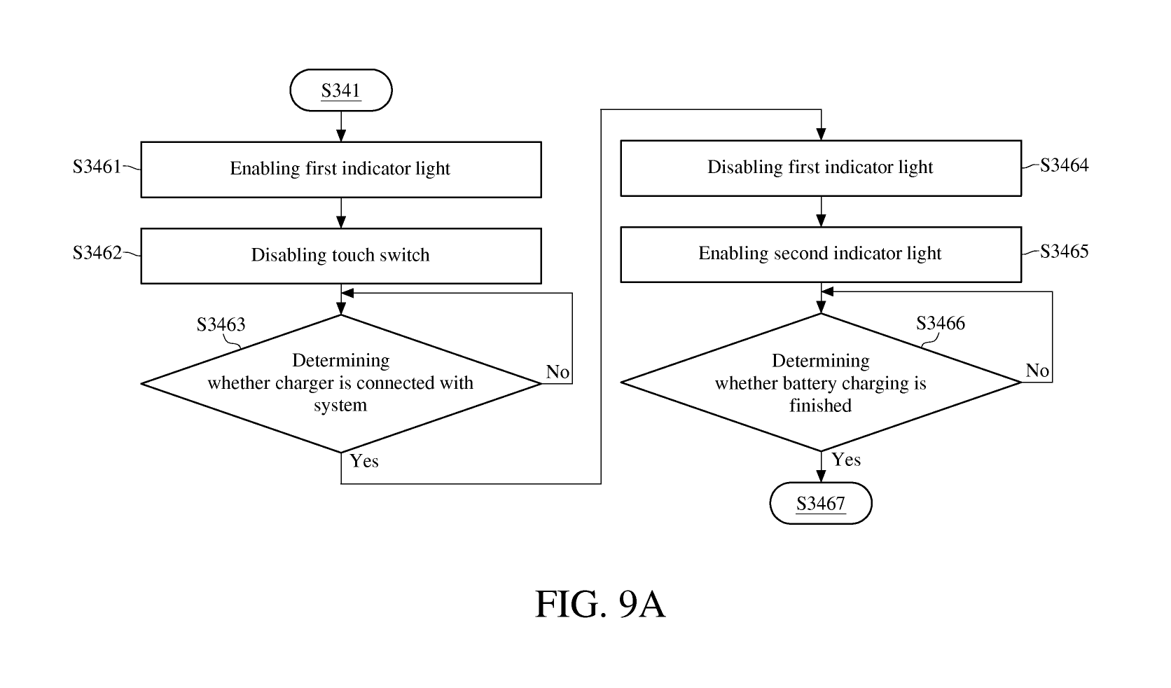

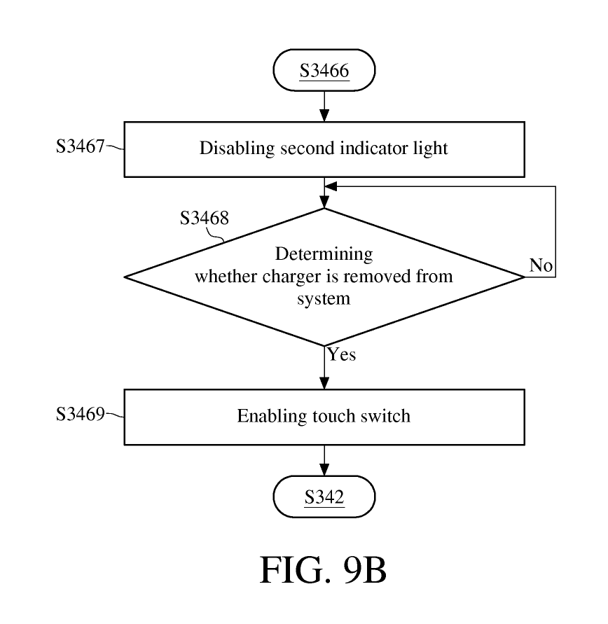

When the working module determines that the electricity of the battery 150 is less than or equal to the first default threshold value, the charging procedure is executed (the step S348). Please refer to FIG. 9A and FIG. 9B together. FIG. 9A is a flowchart of part of the step S348 according to an embodiment of the present invention. FIG. 9B is a flowchart of part of the step S348 following FIG. 9A according to an embodiment of the present invention. The charging procedure includes enabling a first indicator light (the step S3461) and disabling a touch switch (the step S3462), and determining whether a charger is connected with the system (the step S3463). In the present embodiment, the charger is but not limited to an external direct current (DC) power source or a power supply. When the charger is connected with the system, the following steps are executed: disabling the first indicator light (the step S3464), enabling the second indicator light (the step S3465), determining whether charging the battery 150 is finished (the step S3466), and executing the following steps when charging the battery 150 is finished: disabling the second indicator light (the step S3467), and determining whether the charger is removed from the system (the step S3468), and enabling the touch switch when the charger is removed from the system (the step S3469).

For example, when the electricity of the portable thermal therapy apparatus is less than the half of the electricity in the battery 150, the first indicator light of the device is lit, and the touch switch of the device is disabled. In other words, the touch activation function of the device does not have any effect and the user is not able to activate the device. Next, after the user plugs the charger into the device, the first indicator light is extinguished, and the second indicator light is lit, which means the device is charging normally. When the battery 150 is fully charged, the second indicator light of the device is extinguished, which means the charging process is finished. After the charger is unplugged from the device, the function of the touch switch is resumed, and the charging process is finished. In the present embodiment, the second indicator light is both the system working indicator light and the charging indicator light. In other words, when the charger is not plugged, lightening the second indicator light means the system is working normally.

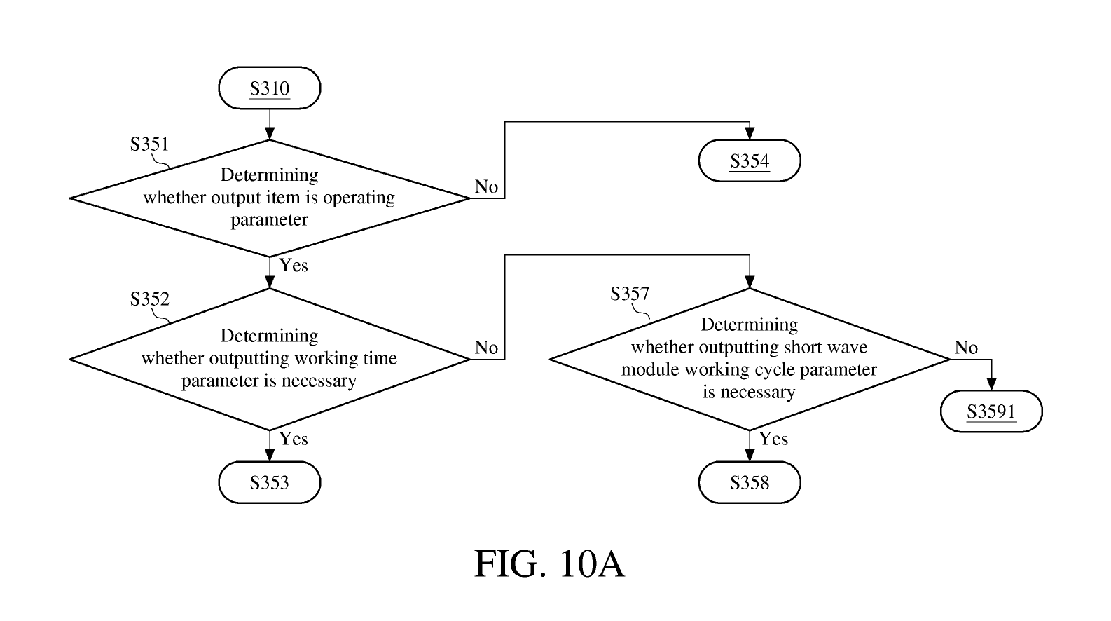

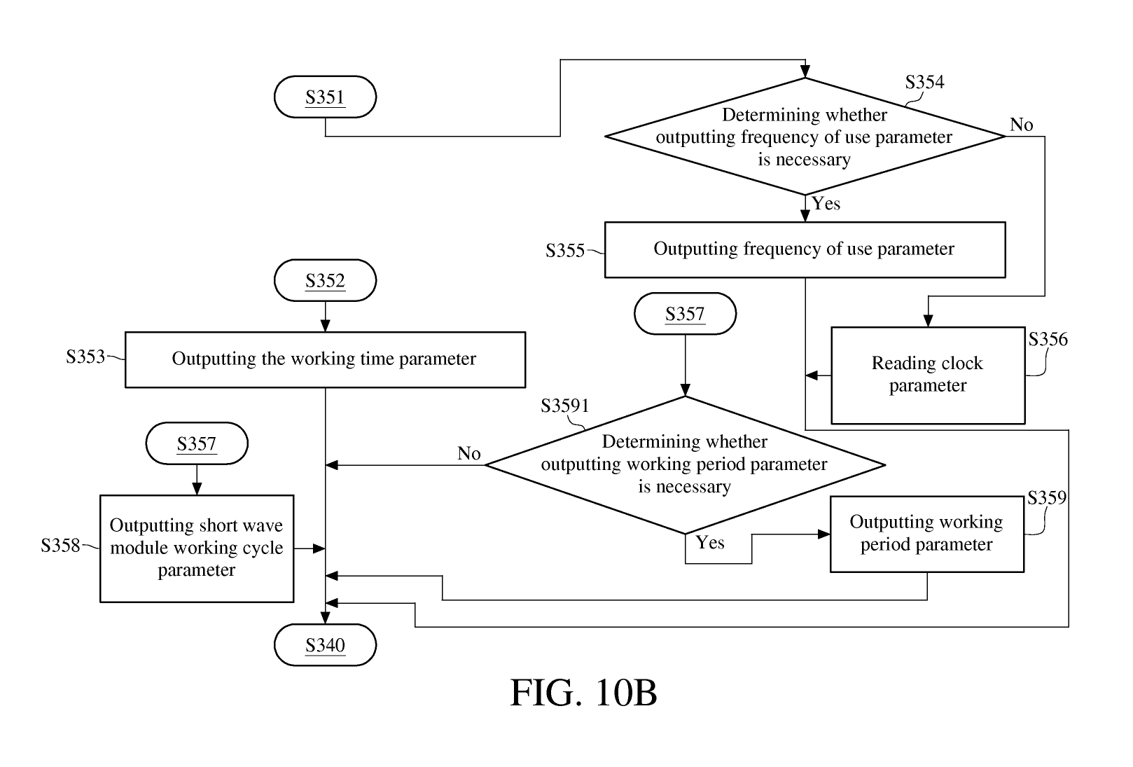

When the working module determines that the operating mode is not the input mode, an output mode is executed. Please refer to FIG. 10A and FIG. 10B together. FIG. 10A is a flowchart of part of the step S350 according to an embodiment of the present invention. FIG. 10B is a flowchart of part of the step S350 following FIG. 10A according to an embodiment of the present invention. The procedures of the output mode are similar to the input mode. The difference between the output mode and the input mode is that the input mode is for the portable thermal therapy apparatus to receive the thermal therapy messages from the user for the procedures operating in the device, and the output mode is opposite to the input mode. The output mode outputs the recorded messages stored in the portable thermal therapy apparatus to the user. The related device mentioned above is not further described hereinafter.

The output mode includes the following steps. First, the working module determines whether an output item is the operating parameter (the step S351). The following steps are executed when the output item is the operating parameter: determining whether outputting a working time parameter is necessary (the step S352) and outputting the working time parameter when outputting the working time parameter is necessary (the step S353). The following steps are executed when outputting the working time parameter is not necessary: determining whether outputting a short wave module working cycle parameter is necessary (the step S357), and outputting the short wave module working cycle parameter when outputting the short wave module working cycle parameter is necessary (the step S358), and determining whether outputting a working period parameter is necessary when outputting the short wave module working cycle parameter is not necessary, and outputting the working period parameter when outputting the working period parameter is necessary (the step S359).

When the output item is not the operating parameter, the working module executes the following steps: determining whether outputting the frequency of use parameter is necessary (the step S354), and outputting the frequency of use parameter when outputting the frequency of use parameter is necessary (the step S355), and reading a clock parameter when outputting the frequency of use parameter is not necessary (the step S356).

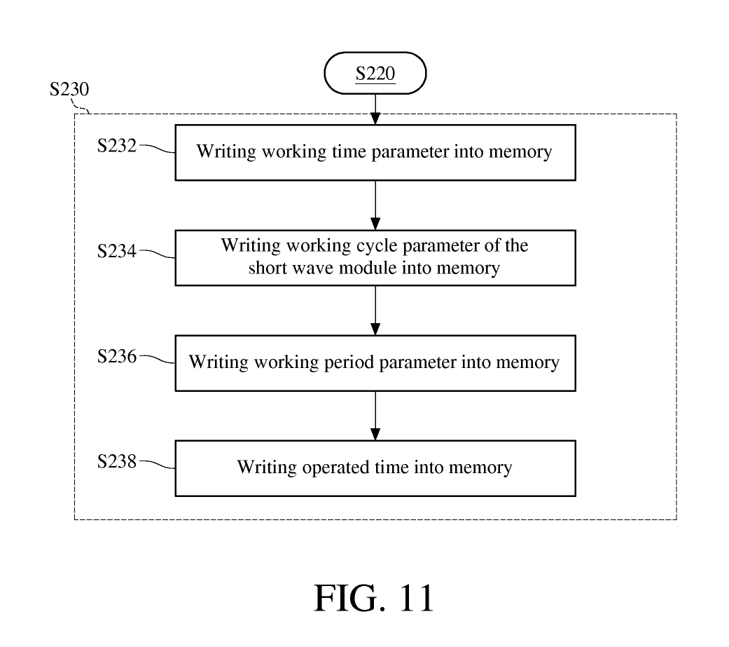

Finally, when the working module finishes the thermal therapy procedure set by the user, please refer to FIG. 11 for the step of recording the operating mode into an operating log message. FIG. 11 is a flowchart of the step S230 according to an embodiment of the present invention. The step includes writing the working period parameter into a memory (the step S232), and writing the short wave module working cycle parameter into the memory (the step S234), and writing the working period parameter into the memory (the step S236), and writing the operated time into the memory (the step S238). In the steps S232 to S238, the working module records the settings of the user, executed thermal therapy procedure, and the practical operating time into the memory after the working module finishes all the thermal therapy procedure. In the present embodiment, the memory is but not limited to the memory unit. When the user uses the portable thermal therapy apparatus next time, the user or the professional reads the previous information of usage from the memory for setting the therapy in the future.

In summary, the portable thermal therapy apparatus of the present invention receives the operating command, and the operating command includes at least an operating parameter. The operating procedure is executed according to the operating command, and the operating procedure at least includes the operating mode. The working module records the operating mode into the operating log message to the memory. Therefore, the portable thermal therapy apparatus of the present invention provides portability, loading the therapy in advance, and the function of recording operating log message, so that the portable thermal therapy apparatus is more convenient for the user.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments of the invention. It is intended, therefore, that the specification and examples be considered as exemplary only, with a true scope and spirit of the invention being indicated by the following claims and their full scope of equivalents.

What is claimed is:

1. An operating method for a portable thermal therapy apparatus, comprising:
receiving an operating command, the operating command at least having an operating parameter;
executing an operating procedure according to the operating command, the operating procedure at least comprising an operating mode; and
recording the operating mode into an operating log message.

2. The operating method for a portable thermal therapy apparatus of claim 1, wherein the step of receiving an operating command, the operating command at least having an operating parameter, comprises:
receiving an input command;
determining whether the operating mode is an input mode; and
executing the following steps when the operating mode is the input mode:
determining whether an input item is the operating parameter;
executing a writing operating parameter procedure when the input item is the operating parameter; and
executing the operating mode.

3. The operating method for a portable thermal therapy apparatus of claim 1, wherein the step of executing the operating procedure according to the operating command, the operating procedure at least having the operating mode, comprises:
determining whether the electricity of a battery is greater than a first default threshold value; and
executing the following steps when the electricity of the battery is greater than a first default threshold value:
enabling a second indicator light;
starting counting an operating time;
executing a running procedure;
determining whether an operated time is longer than a first setting time; and
executing the following steps when the operated time is longer than the first setting time:
disabling a short wave module;
disabling the second indicator light; and
recording the operating mode into an operating log message.

4. The operating method for a portable thermal therapy apparatus of claim 3, wherein the running procedure comprises:
enabling the short wave module;
enabling a heater module;
determining whether the temperature of a temperature sensing module is greater than a first default temperature; and
disabling the heater module when the temperature of the temperature sensing module is greater than the first default temperature.

5. The operating method for a portable thermal therapy apparatus of claim 4, wherein when the electricity of the battery is less than or equal to the first default threshold value, a charging procedure is executed.

6. The operating method for a portable thermal therapy apparatus of claim 5, wherein the charging procedure comprises:
enabling a first indicator light;
disabling a touch switch;
determining whether a charger is connected with the system; and
executing the following steps when the charger is connected with the system:
disabling the first indicator light;
enabling the second indicator light;
determining whether a battery charging is finished; and
executing the following steps when the battery charging is finished:
disabling the second indicator light;
determining whether the charger is removed from the system; and
enabling the touch switch when the charger is removed from the system.

7. The operating method for a portable thermal therapy apparatus of claim 6, wherein the writing operating parameter procedure comprises:
determining whether writing a working time parameter is necessary;
writing the working time parameter when writing the working time parameter is necessary; and
executing the following steps when writing the working time parameter is not necessary:
determining whether writing a short wave module working cycle parameter is necessary;
writing the short wave module working cycle parameter when writing the short wave module working cycle parameter is necessary; and
writing the working period parameter when writing the short wave module working cycle parameter is not necessary.

8. The operating method for a portable thermal therapy apparatus of claim 7, wherein when the input item is not the operating parameter, a writing system parameter procedure is executed.

9. The operating method for a portable thermal therapy apparatus of claim 8, wherein the writing system parameter procedure comprises the following steps:
determining whether zeroing a frequency of use parameter is necessary;
zeroing the frequency of use parameter when zeroing the frequency of use parameter is necessary; and
writing a clock parameter when zeroing the frequency of use parameter is not necessary.

10. The operating method for a portable thermal therapy apparatus of claim 1, wherein when the portable thermal therapy apparatus is not in the input mode, an output mode is executed.

11. The operating method for a portable thermal therapy apparatus of claim 10, wherein the output mode comprises the following steps:
determining whether an output item is the operating parameter; and
executing the following steps when the output item is the operating parameter:
determining whether outputting a working time parameter is necessary;
outputting the working time parameter when outputting the working time parameter is necessary; and
executing the following steps when outputting the working time parameter is not necessary:
determining whether outputting a short wave module working cycle parameter is necessary;
outputting the short wave module working cycle parameter when outputting the short wave module working cycle parameter is necessary; and
determining whether outputting a working period parameter is necessary when outputting the short wave module working cycle parameter is not necessary, and outputting the working period parameter when outputting the working period parameter is necessary.

12. The operating method for a portable thermal therapy apparatus of claim 11, wherein when the output item is not the operating parameter, the following steps are executed:
determining whether outputting a frequency of use parameter is necessary, and outputting the frequency of use parameter when outputting the frequency of use parameter is necessary; and
reading a clock parameter when outputting the frequency of use parameter is not necessary.

13. The operating method for a portable thermal therapy apparatus of claim 1, wherein the step of recording the operating mode into the operating log message comprises:
writing the working time parameter, the working cycle parameter of the short wave module, the working period parameter, and the operated time into a memory.

14. A portable thermal therapy apparatus, comprising:
an input unit for receiving an operating command, the operating command at least comprising an operating parameter;
a processor and a memory unit for executing an operating procedure according to the operating command and recording the operating mode into an operating log message, the operating procedure at least comprising an operating mode;
a power management unit for executing a charging procedure;
a thermal therapy module for executing a running procedure;
a battery for providing the portable thermal therapy apparatus with necessary electricity; and
an external power source for providing a direct current (DC) power source to the power management unit of the portable thermal therapy apparatus.

15. The operating method for a portable thermal therapy apparatus of claim 14, wherein the thermal therapy module comprises:
a short wave module for generating a diathermy;
a heater module for generating a heat source; and
a temperature sensing module for monitoring the heater module.

16. The operating method for a portable thermal therapy apparatus of claim 14, wherein the step of receiving the operating command, the operating command at least having the operating parameter, comprises:
receiving an input command;
determining whether the operating mode is an input mode; and
executing the following steps when the operating mode is the input mode:
determining whether an input item is the operating parameter;
executing a writing operating parameter procedure when the input item is the operating parameter; and
executing the operating mode.

17. The operating method for a portable thermal therapy apparatus of claim 14, wherein the step of executing the operating procedure according to the operating command, the operating procedure at least comprising the operating mode, comprises:
determining whether the electricity of the battery is greater than a first default threshold value; and
executing the following steps when the electricity of the battery is greater than the first default threshold value:
enabling an operation indicator light;
starting counting an operating time;
executing a running procedure;
determining whether an operated time is longer than a first setting time; and
recording the operating mode into an operating log message when the operated time is longer than a first setting time.

18. The operating method for a portable thermal therapy apparatus of claim 17, wherein the running procedure comprises:
enabling a short wave module;
enabling a heater module;
determining whether the temperature of a temperature sensing module is greater than the temperature of a first default temperature; and
disabling the heater module when the temperature of the temperature sensing module is greater than the temperature of the first default temperature.

19. The operating method for a portable thermal therapy apparatus of claim 18, wherein when the electricity of the battery is less than or equal to the first default threshold value, a charging procedure is executed.

20. The operating method for a portable thermal therapy apparatus of claim 19, wherein the charging procedure comprises:
enabling a first indicator light;
determining whether a charger is connected with the system; and
executing the following steps when the charger is connected with the system:
disabling the first indicator light;
determining whether a battery charging process is finished; and
executing the following steps when battery charging process is finished:
enabling a second indicator light;
determining whether the charger is removed from the system; and
disabling the second indicator light when the charger is removed from the system.

21. The operating method for a portable thermal therapy apparatus of claim 20, wherein the writing operating parameter procedure comprises:
determining whether writing a working time parameter is necessary;
writing the working time parameter when writing the working time parameter is necessary; and
executing the following steps when writing the working time parameter is not necessary:
determining whether writing a short wave module working cycle parameter is necessary;
writing the short wave module working cycle parameter when writing the short wave module working cycle parameter is necessary; and
writing the working period parameter when writing the short wave module working cycle parameter is not necessary.

22. The operating method for a portable thermal therapy apparatus of claim 21, wherein when the input item is not the operating parameter, a writing system parameter procedure is executed.

23. The operating method for a portable thermal therapy apparatus of claim 22, wherein the writing system parameter procedure comprises the following steps:
determining whether zeroing a frequency of use parameter is necessary;
zeroing the frequency of use parameter when zeroing the frequency of use parameter is necessary; and
writing a clock parameter when zeroing the frequency of use parameter is not necessary.

24. The operating method for a portable thermal therapy apparatus of claim 14, wherein when the operating mode is not the input mode, an output mode is executed.

25. The operating method for a portable thermal therapy apparatus of claim 24, wherein the output mode comprises the following steps:
determining whether an output item is the operating parameter; and
executing the following steps when the output item is the operating parameter:
determining whether outputting a working time parameter is necessary;
outputting the working time parameter when outputting the working time parameter is necessary; and
executing the following steps when outputting the working time parameter is not necessary:
determining whether outputting a short wave module working cycle parameter is necessary;
outputting the short wave module working cycle parameter when outputting the short wave module working cycle parameter is necessary; and
outputting a working period parameter when outputting the short wave module working cycle parameter is not necessary.

26. The operating method for a portable thermal therapy apparatus of claim 25, wherein when the output item is the operating parameter, the following steps are executed:
determining whether outputting a frequency of use parameter is necessary and outputting the frequency of use parameter when outputting the frequency of use parameter is necessary; and
reading a clock parameter.

27. The operating method for a portable thermal therapy apparatus of claim 26, wherein the step of recording the operating mode into the operating log message comprises:
writing the working time parameter, the working cycle parameter of the short wave module, the working period parameter, and the operated time into a memory.
